(19) United States
(12) Patent Application Publication
(10) Pub. No.: US 2002/0037260 A1
(43) Pub. Date: Mar. 28, 2002

(54) Compositions for treating biofilm

(76) Inventors: John A. Budny, Westlake Village, CA (US); Matthew J. Budny, Westlake Village, CA (US)

Correspondence Address: COLIN P ABRAHAMS, 5850 CANOGA AVENUE, SUITE 400, WOODLAND HILLS, CA, 91367

(21) Appl. No.: 09/876,248

(22) Filed: Jun. 6, 2001

Related U.S. Application Data
(63) Continuation-in-part of application No. 09/587,818, filed on Jun. 6, 2000.
Application No. 09/587,818 is a continuation-in-part of application No. 09/249,674, filed on Feb. 12, 1999, now Pat. No. 6,159,447.
Application No. 09/249,674 is a continuation-in-part of application No. 08/951,393, filed on Oct. 16, 1997, now Pat. No. 5,871,714.

(51) Int. Cl. A61K 38/48; A61K 7/16; A61K 7/28; A61K 38/16; A61K 31/496

(52) U.S. Cl. 424/49; 424/50; 424/94.63; 514/8; 514/253.08

(57) ABSTRACT

A composition for treating a biofilm comprises a first anchor enzyme component to degrade biofilm structures and a second anchor enzyme component having the capability to act directly upon the bacteria for a bactericidal effect.

### CROSS REFERENCE TO RELATED APPLICATIONS

This application is a continuation-in-part of U.S. application Ser. No. 09/587,818 filed Jun. 06, 2000, which is a continuation-in-part of U.S. application Ser. No. 09/249,674 filed Feb. 12, 1999 (issued as U.S. Pat. No. 6,159,447 on Dec. 12, 2000), which is a continuation-in-part of U.S. application Ser. No. 08/951,393 filed Oct. 16, 1997 (issued as U.S. Pat. No. 5,871,714 on Feb. 16, 1999), both of which are incorporated herein by reference.

### FIELD AND BACKGROUND OF THE INVENTION

Standard chemical analyses, traditional microscopic methods as well as digital imaging techniques such as confocal scanning laser microscopy, have transformed the structural and functional understanding of biofilms. Investigator using these techniques have a clearer understanding of biofilm-associated microorganism cell morphology and cellular functions.

Biofilms are matrix-enclosed accumulations of microorganisms such as bacteria (with their associated bacteriophages), fungi, protozoa and viruses that may be associated with these elements. While biofilms are rarely composed of a single cell type, there are common circumstances where a particular cellular type predominates. The non-cellular components are diverse and may include carbohydrates, both simple and complex, proteins, including polypeptides, lipids and lipid complexes of sugars and proteins (lipopolysaccharides and lipoproteins).

For the most part, the unifying theme of non-cellular components of biofilms is its backbone. In virtually all known biofilms, the backbone structure is carbohydrate or polysaccharide-based. The polysaccharide backbone of biofilms serves as the primary structural component to which cells and debris attach. As the biofilm grows, expands and ages along biologic and non-biologic surfaces in well-orchestrated enzymatic synthetic steps, cells (planktonic) and non-cellular materials attach and become incorporated into the biofilm. The growing biofilm not only attracts living cells; it also captures debris, cell fragments, insoluble macromolecules and other materials that add to the layer upon the polysaccharide backbone. In this fashion, layering continues and is repeated so that the initial layers of the polysaccharide backbone, become buried or embedded in the biofilm. As the biofilm ages, there are layers upon layers of polysaccharide backbone with the attendant cells, debris and insoluble macromolecular structures.

Biofilms are the most important primitive structure in nature. In a medical sense, biofilms are important because the majority of infections that occur in animals are biofilm-based. Infections from planktonic bacteria, for example, are only a minor cause of infectious disease. In industrial settings, biofilms inhibit flow-through of fluids in pipes, clog water and other fluid systems and serve as reservoirs for pathogenic bacteria and fungi. Industrial biofilms are an important cause of economic inefficiency in industrial processing systems.

Biofilms are prophetic indicators of life-sustaining systems in higher life forms. The nutrient-rich, highly hydrated biofilms are not just layers of planktonic cells on a surface; rather, the cells that are part of a biofilm are a highly integrated “community” made up of colonies. The colonies, and the cells within them, express exchange of genetic material, distribute labor and have various levels of metabolic activity that benefits the biofilm as a whole.

Planktonic bacteria, which are metabolically active, are adsorbed onto a surface as the initial step in the colonization process. Once adsorbed onto a surface, the initial colonizing cells undergo phenotypic changes that alter many of their functional activities and metabolic paths. For example, at the time of adhesion, Pseudomonas aeruginosa (P. aeruginosa) shows up regulated algC, algD, algU etc. genes which control the production of phosphomanomutase and other pathway enzymes that are involved in alginate synthesis which is the exopolysaccharide that serves as the polysaccharide backbone for Pseudomonas aeruginosa biofilm. As a consequence of this phenotypic transformation, as many as 30 percent of the intracellular proteins are different between planktonic and sessile cells of the same species.

In summary, planktonic cells adsorb onto a surface, experience phenotypic transformations and form colonies. Once the colonizing cells become established, they secrete polysaccharides that serves as the backbone for the growing biofilm. While the core or backbone of the biofilm is derived from the cells themselves, components e.g., lipids, proteins etc, from other sources become part of the biofilm. Thus a biofilm is heterogeneous in its total composition, creating diffusion gradients for materials and molecules that attempt to penetrate the biofilm structure.

Biofilm-associated or sessile cells predominate over their planktonic counterparts. Not only are sessile cells physiologically different from planktonic members of the same species, there is phenotypic variation within the sessile subsets or colonies. This variation is related to the distance a particular member is from the surface onto which the biofilm is attached. The more deeply a cell is embedded within a biofilm i.e., the closer a cell is to the solid surface to which the biofilm is attached or the more shielded or protected a cell is by the bulk of the biofilm matrix, the more metabolically inactive the cells are. The consequences of this variation and gradient create a true collection of communities where there is a distribution of labor, creating an efficient system with diverse functional traits.

Biofilm structures cause the reduced response of bacteria to antibiotics and the bactericidal consequences of antimicrobial and sanitizing agents. Antibiotic resistance and persistent infections that are refractory to treatments are a major problem in bacteriological transmissions, resistance to eradication and ultimately pathogenesis. While the consequences of bacterial resistance and bacterial recalcitrance are the same, there are two different mechanisms that explain the two processes.

The use of enzymes in degrading biofilms is not new. Compositional patents as well as published scientific literature support the concept of using enzymes to degrade, remove and destroy biofilms. However, the lack of consistency in results and the inability to retain the enzymes at the site where their action is required has limited their widespread use.

As an alternative to enzymes, harsh chemicals, elevated temperatures and vigorous abrasion procedures are used. There are conditions, however, where these non-enzymatic approaches cannot be used e.g., caustic- and acidic-sensitive environments, temperature or abrasion sensitive components that are associated with the biofilm and dynamic fluid action. When a biofilm is growing in an area where there is a constant fluid flow, the agents that remove biofilms are flushed away before they can carry our their desired function. This is particularly true for medical situations where aggressive sterilization procedures cannot be carried out and there is a desired fluid flow.

Harsh treatments employed to control biofilms in certain situations (extreme heat, pH conditions, abrasion, etc.) are often inappropriate for their use in biologic systems. Biofilms in the oral cavity, biofilms associated with implanted devices and infections that occur in the respiratory, alimentary and vaginal tracts or in eyes, ears etc. are particularly suited for an enzymatic treatment. There are also specific disease conditions, such as pneumonia and cystic fibrosis which are bacteria-based and occur in the lung, that would benefit from an enzymatic treatment, but only if the enzymes could be retained at the site long enough to fully realize their therapeutic actions.

Biofilm growth and the proliferation of infections in biologic systems are particularly sensitive to fluid-flow dynamics. Specific organs where infections occur e.g. eyes, oral cavity, gastrointestinal tract, vaginal tract, lungs etc., fluid and mucus flows are an integral part of the system's normally functioning mode. Biofilm control in these environments demand non-harsh measures, such as enzymatic destruction and/or removal; however, due to fluid-flow characteristics in these systems, a method must employed to prevent the enzymes from being swept away by fluid flow. The present invention provides a method of retaining the enzymes in close proximity to the biofilm where it is intended to function.

It is also desirable to not only be able to degrade a biofilm within a biologic system, but also to be able to have a direct effect on the bacterial cells that are released as the biofilm is undergoing degradation. The combination of biofilm degradation and agents that directly affect bacterium is also not a new strategy. However, not infrequently in an open system, the same forces that flush or sweep away the biofilm degrading enzymes also flush bactericidal agents so that they cannot act directly upon bacteria unless there is a chance meeting between the agent and a planktonic bacterium.

### SUMMARY OF THE INVENTION

According to one aspect of the invention, there is provided a composition for treating a biofilm structure comprising: a first enzyme-anchor component comprising an enzyme selected for its ability to degrade the biofilm structure and an anchor selected for its ability to attach to a surface on or proximal the biofilm structure to increase retention time, and a second enzyme-anchor component comprising an enzyme selected for its ability to act directly upon bacteria from the biofilm structure for a bactericidal effect thereon and an anchor selected for its ability to attach to a surface on or proximal the biofilm structure.

Gene transfer between bacteria in a biofilm may facilitate resistance of the bacteria to antibiotics and/or antimicrobial agents. Further, antibiotic/antimicrobial recalcitrance may occur when (a) the biofilm structures present a barrier to penetration of antibiotics and antimicrobial agents and a protective shroud to physical agents such as ultraviolet radiation and/or (b) the biofilm also acts as a barrier to nutrients that are necessary for normal metabolic activity of the bacteria. Thus, the nutrient-limited bacteria are in a reduced state of metabolic activity, which make them less susceptible to chemical and physical agents because the maximal effects of these killing agents are achieved only when the bacteria are in a metabolically active state.

With any of the possible mechanistic explanations for resistance or recalcitrance, removal or disruption of the biofilm is a mandatory requirement. Stripping away of the biofilm components e.g., the polysaccharide backbone with the accumulated debris accomplishes several objectives: 1) reduced opportunity for gene transfer; 2) increased penetration of chemical and physical agents; and 3) increased free-flow of nutrients which would elevate the metabolic activity of the cells and make them more susceptible to chemical and physical agents. Furthermore, removal or disruption of the biofilm will free cells from a sessile state to make them planktonic which also increases their susceptibility to chemical and physical agents.

Biofilm structures occur in animals as an infection or in an environment that is not living such as a medical device or implant that is in contact with living tissue, or in an industrial setting. In all cases, the biofilm impedes the treatment and removal of the organisms that cause the biofilm. In the case of animal infections, antibiotics and the host's own immune responses are less effective. In an industrial setting, harsh treatments are necessary and often these treatments either do not work completely or they have to be repeated.

In order to destroy established biofilms, with various levels of embedded cells, the disruption, fragmentation and removal of the biofilm is necessary. This can be accomplished, under limited circumstances, with physical means e.g., abrasion methods, sonication, electrical charge stimulation, detergent and enzymatic. There are obvious drawbacks to any one method, precluding a universal method or approach. However, the common trait of all of these methods lies in their focus on the biofilm structure and not the living cells within the biofilm.

If, by any one of the methods, the structure of the biofilm is altered or disturbed, a secondary, complementary attack on the living cells within the biofilm can be made with antibiotics, antibacterials and antimicrobial agents.

One aspect of the invention lies in two areas, both of which may operate independently, but when combined, effectively remove biofilms and prevent their reestablishment. The first area is the removal of the biofilm structure in an orderly and controlled manner using enzymes. The second area employs agents, such as enzymes, antimicrobial agents, antibiotics etc. to kill the bacteria that were part of the biofilm structure.

During the removal or dismantling of the biofilm structure, especially the polysaccharide backbone, cells within the biofilm become more susceptible to the bactericidal action of antibacterials, antimicrobials, antibiotics, sanitizing agents and host immune responses. As the biofilm is removed, some cells within the biofilm are liberated and become planktonic; others, however, remain sessile but are more vulnerable to being killed because the protective quality of the biofilm, essentially the outer layers that shield or protect the embedded cells, is reduced.

One aspect of the invention provides at least one enzyme whose specificity includes its ability to degrade polysaccharide backbone structure(s) of a biofilm produced by bacterial strain(s). While this polysaccharide-degrading enzyme is hydrolytic, it is found in four major classifications, as follows with examples:

Carboxylic Ester Hydrolases (EC 3.1.1.-)

Pectin Esterase (EC 3.1.1.11); Lactonase (EC 3.1.1.25); Acetylesterase (EC 3.1.1.6), et al.

Sulfuric Ester Hydrolases (EC 3.1.6.-)

Glycosulfatase (EC 3.1.6.3); Chondroitinsulfatase (EC 3.1.6.4); Cellulase polysulfatase (EC 3.1.6.7); Chondro-n-sulfatase (EC 3.1.6.n); Disulfoglucosamine-6-sulfatase (EC 3.1.6.11); N-acetylglucosamine-6-sulfatase (EC 3.1.6.14 ) et al.

Glycosidases (EC 3.2.-.-)

Amylase, α and β (EC 3.2.1.1 and 2); Exo-1,4-α-glucosidase (EC 3.2.1.3); Cellulase (EC 3.2.1.4); Oligo-1,6-glucosidase (EC 3.2.1.10); Dextranase (EC 3.2.1.11); Pectin depolymerase (EC 3.2.1.15); Lysozyme (EC 3.2.1.17); Nuraminidase (EC 3.2.1.18); β-galactosidase (EC 3.2.1.23); β-fructofuranosi-dase (EC 3.2.1.26); β-N-acetyl-D-hexosaminidase (EC 3.2.1.30); β-D-glucuroni-dase (EC 30 3.2.1.31); Xylanase (EC 3.2.1.32); Mucinase (EC 3.2.1.35) [Hyaluronidase (EC 3.2.1.35)]; Pullulanase (EC 3.2.1.41); Sucrose α-glucosidase (EC 3.2.1.48); Mutanase (Glucan endo-1,3-α-glucosidase (EC 3.2.1.59); 2,6-β-fructan 6-levanbiohydrolase (EC 3.2.1.64); Levanase (EC 3.2.1.65); Fructan β-fructosidase (EC 3.2.1.80); Galactohydrolase (capsular) (EC 3.2.1.87); Sphinganase; Gellanase; β-galactanase et al.

Lyases Acting on Polysaccharides (EC 4.2.2.-)

Pectin lyase (EC 4.2.2.10); Alginate lyase (EC 4.2.2.3); Exopolygalacturonic acid lyase (EC 4.2.2.9); Hyaluronate lyase (EC 4.2.2.1; EC 4.2.99.1); Pectate lyase (EC 4.2.2.2); Polysaccharide depolymerase; Emulsan depolymerase; Guluronan lyase (EC 4.2.2.11); Heparin lyase (EC 4.2.2.7); Heparitin-sulfate lyase (EC 4.2.2.8); Non-specific polysaccharide depolymerases et al.

Additionally, polysaccharide degrading enzymes can be obtained from bacteriophages. While these depolymerases, when delivered by the bacteriophage, degrade the polysaccharide in the capsule surrounding the bacterium, they are also capable of degrading the polysaccharides that make up the biofilm backbone.

Attached to the enzyme(s), either through chemical synthetic procedures or recombinant technology, are one or more moieties that have the capability of binding either reversibly (non-covalently) or irreversibly (covalent bonded) to a surface near the biofilm or the biofilm itself. Collectively, these moieties are called anchors. The moieties selected to serve as anchors can be agents or molecular species known to have an affinity for the biofilm or the surfaces near the biofilm or known binding domains. Examples of these types of anchors are listed below. The listing is not intended to be a complete list; rather, the listed examples serve to illustrate the entire class. Finally, the search for anchors can be accomplished with High Throughput Screening (HTS) of a biofilm of either known or unknown composition with various molecular entities using a suitable assay to determine which materials have an affinity for the biofilm or its surrounding surface.

These two properties: 1. an enzyme; and 2. a binding component that is connected to the enzyme, are directed at the degradation of the biofilm backbone structure.

Moieties with a Known Affinity for Biofilms

Concanavalin A; Wheat Germ Agglutinin; Other Lectins; Elastase; Amylose Binding Protein;Ricinus communis agglutinin I (RCA I); Dilichos biflorus agglutinin (DBA); Ulex europaeus agglutinin I (UEA I).

Binding Domains from Enzymes

Dextransucrase; Starch-synthesizing enzymes; Cellulose-synthesizing enzymes; Chitin-synthesizing enzymes; Glycogen-synthesizing enzymes; Pectate synthetase; Glycosyl transferase-binding domains (glucan-, mutan-, levan-, Polygalactosyl-synthesizing enzymes; et al.

Certain agents have been described (see U.S. Pat. Nos. 3,309,274; 3,624,219; 4,064,229 and 4,431,628) as indicators or disclosing agents for oral bacterial biofilms. In effect, these agents bind to the biofilm where they can be visualized either by the naked eye or with the aid of a light source with a wavelength that shows the agents color. The purpose of these agents as described in the cited patents is to show location of the biofilm structure.

Since these agents bind to plaque, that property, in and of itself, makes them exceptionally good anchors in the anchor and enzyme complexes. Consequently, any molecular entity whose purpose is to serve as a biofilm disclosing agent can also be used as an anchor for the anchor enzyme complex to retain enzymes at or near a biofilm. Following is a list of examples of biofilm disclosing agents, which are examples of molecules that can serve as anchors. This list is only a selected list of examples and it is not intended to exclude other disclosing agents.

Examples of Biofilm Disclosing Agents

FD&C Red #3 (erythrosin); Amaranth (Brilliant Blue); Synthetic fluorescent dyes; D&C Green #8; D&C Red #s 19, 22 and 28; D&C Yellow #s 7 and 8; Natural fluorescent dyes; Chlorophyll dye; Carotene; FD&C Blue #1; FD&C Green #3; Hercules Green Shade 3; Merbromin; Betacyanines; Betamine; Betanin; Betaxanthines; Vulgaxathin; Ruthenium Red.

Another aspect of the invention consists of two or more hydrolytic enzymes. One enzyme has the specificity to degrade the biofilm's polysaccharide backbone structure of a biofilm; at least one other enzyme is hydrolytic in nature, having the capability to degrade proteins, polypeptides, glycoproteins, lipids, lipid complexes of sugars and proteins (lipopolysaccharides and lipoproteins).

Blends and combinations of enzymes have been used for industrial processing applications and that multiple enzymes, used together, can remove biofilms (Johansen, C., Falholt, P. and Gram, L. “Enzymatic Removal and Disinfections of Bacterial Biofilms.” Applied and Environmental Microbiology, Vol. 93, No. 9, September 1997, p. 3724-3728). As an illustrative example, alginate lyase, pectinase, arabinase, cellulose, hemicullulase, β-glucanase and xylanase, each connected to elastase, with the elastase serving as an anchor to the biofilms, can be used to remove alginate biofilms. Alginate biofilms are ordinarily produced by Pseudomonas aeruginosa and Pseudomonas fluorescens. However, this anchor-enzyme combination described above will effectively remove alginate-based biofilms produced by any bacterial or fungal species, whether they act alone or in combination with one another to create the biofilm.

Another example for removing biofilms produced by Staphylococcus aureus and Staphylococcus epidermidis involves the enzymes β-N-acetylglucosaminidase, pectinase, arabinase, cellulase, hemicellulase, β-glucanase and xylanase each connected to a lectin such as wheat germ agglutinin (WGA) which recognizes and binds to N-acetylglucosamine so that the enzyme can be retained at the site of the biofilm where degradation of the biofilm can occur.

The enzymes capable of degrading proteins and polypeptides are found in classification EC 3.4.-.-. These proteinases include proteolytic enzymes, endopeptidases, peptidyl-peptide hydrolases, serine proteinases, acid proteinases and SH-proteinases. In a universal sense, all of the protein and peptide hydrolysis enzymes cleave the amide linkage between adjacent amino acids in either a polypeptide or protein. Specific examples would include, but not be limited to, peptidases, carboxypeptidase, particle-bound amino peptidase (EC 3.4.11.2), chymotrypsin, trypsin, cathepsin, thrombin, prothrombinase, plasmin, elastase, subtilsin, papain, ficin, asclepain, pepsin, chymosin, collagenase and those enzymes with EC 3.4.99.-, which possess proteinase activity of unknown mechanisms.

Many of the enzymes that hydrolyze glycoproteins (proteoglycans) have not been specifically isolated and characterized. Those proteinases and peptidyl-hydrolyases where the mechanism is not known are initially classified in either EC 3.-.- as hydrolases, most likely falling into EC 3.2.- and EC 3.4.-, and EC 4.2.2.- (Lyases Acting on Polysaccharides).

Examples of Enzymes that Hydrolyze Glycoproteins

Peptidoglycan endopeptidase(hydrolase) (EC 3.4.99.17); Heparin lyase(EC 4.2.2.7); Heparatinase; Chitodextrinase (EC 3.2.1.14); Chondroitin lyase (EC 4.2.2.4; EC 4.2.2.5); Muramindase (EC 3.2.1.17); ; N-Acetylmuramidase; Sialidase/Neuraminidase (EC 3.2.1.18); β-N-Acetylhexosaminidase (EC 3.2.1.52); α-N-Acetylhexosaminidase; β-N-Acetylglucosaminidase (EC 3.2.1.30); Hyaluronoglucosidase (EC 3.2.1.35); Hyaluronoglucuronidase (EC 3.2.1.36); β-N-Acetylgalactosaminidase (EC 3.2.1.53); β-Aspartylacetylglucosaminidase (EC 3.2.2.1) et al.

Enzymes capable of attacking lipids are called lipases in a broad sense and are classified as EC 3.1.-.-. Specific examples include, but are not limited to: Hexoselipase; Galactolipase (EC 3.1.1.26); Diacylglycerol lipase (lipoprotein lipase) (EC 3.1.1.34); Glucosylceramidase (EC 3.2.1.45); Galactosylceramidase (EC 3.2.1.46); Galactosylgalactosylglucosylceramidase (EC 3.2.1.47); Cerebroside sulfatase (EC 3.1.6.8) et al.

Attached to the enzymes, either individually or collectively as a single unit through chemical synthetic procedures or recombinant technology, are one or more moieties that have the capability of binding either reversibly (non-covalently) or irreversibly (covalent bonded) to a surface near the biofilm or the biofilm itself. This aspect is directed at the degradation and removal of the biofilm backbone structure along with any other materials that may be associated with the backbone, which collectively constitute the entire biofilm. Examples of anchors have been described above.

Still another aspect of the invention consists of two or more enzymes, wherein at least one enzyme has the capability of degrading a biofilm structure produced by a bacterial strain, or a mixed combination of various strains, and the other enzymes(s) has (have) the capability of acting directly upon the bacteria, causing lysis of the bacterial cell wall. One or more moieties are attached to the enzymes, forming either a single unit or multiple units. The moieties are attached to the enzymes either through chemical synthetic procedures or recombinant technology to give the enzyme moiety the capability of binding either reversibly (non-covalently) or irreversibly (covalently bonded) to a surface near the biofilm or the biofilm itself. The purpose of this multi-enzyme system is directed at the degradation and removal of the biofilm with the contemporaneous bactericidal consequences for bacteria that were embedded in the biofilm's structure and which have become exposed due to the action of the biofilm-degrading enzyme(s).

Lysozyme has long been known to have bactericidal activity by destroying the bacterial cell wall, freeing cell wall components which leads to cell lysis. Anchored lysozyme, along with anchored polysaccharide-degrading enzyme(s), can be used in concert to remove the polysaccharide backbone of a biofilm and then lyse the resident bacteria in a stepwise fashion. In a specific example of the removal of oral biofilms, lysozyme can be connected to amylase binding protein or the glucan binding domain, either by coupling the lysozyme to the selected anchor or through a recombinant synthesis. The consequence of this combination is that the polysaccharide backbone is removed and the embedded bacteria are killed through cell lysis at the same time.

Lysozyme can be used in the treatment and removal of other biofilms along with the resident bacteria, that may exist outside of the oral cavity. For biofilms produced by Pseudomonas aeruginosa and Pseudomonas fluorescens, lysozyme can be anchored with elastase and used in conjunction with any one of the following biofilm-degrading enzymes: alginate lyase, pectinase, arabinase, cellulase, hemicullulase, β-glucanase and/or xylanase, each connected to elastase or some other suitable anchor.

This multi-enzyme, dual functionality for treating and eliminating biofilms can be used for any microorganism that produces a biofilm e.g., fungi.

Examples of Enzymes that have the Capability to Kill Bacteria:

Lysozyme (EC 3.2.1.17); Mucinase (EC 3.2.1.35); Neuraminidase (EC 3.2.1.18); Keratanase (EC 3.2.1.103); Capsular polysaccharide galactohydrolase (EC 3.2.1.87); Glycoside hydrolase (EC 3.2.1.-); Chondroitin ABC lyase (EC 4.2.2.4); Heparatinase; Heparin lyase (EC 4.2.2.4); Glycosaminoglycan (EC 4.2.2.-); Pectate lyase (EC 4.2.2.2); Peptidoglycan hydrolase (Lysostaphin) (EC 3.4.99.17); Any bacteriophage polysaccharide depolymerase; holin enzymes; lysin; endolysin; lysostaphin et al.

Many bacteriophage enzymes require specific proteins that assist in the penetration of the lytic enzyme into the bacterial cell wall. These proteins, called holins, may be associated with the genes that encode the lytic enzymes. Holins are believed to assist the lytic enzymes to gain access to the components of the bacterial cell wall that serve as a substrate for the enzyme. These holing proteins may be enzymes themselves.

Another aspect of the invention consists of two sets of enzymes, the first being one or more enzymes with the appropriate anchor attached to the enzyme(s) for the purpose of degrading the biofilm structure, the second set of enzymes also being connected to anchor molecules whose function is to generate active oxygen to directly attack and kill bacteria that are exposed during the process of the degradation and removal of the biofilm.

Any enzymes in EC 3.-.-.- and EC 4.-.-.- may be used, including those previously mentioned, which have the capability to degrade biofilm structures, plus those enzymes that can produce active oxygen. Specifically, the enzymes that can produce active oxygen are oxidoreductases, found in EC 1.-.-.-. Examples of such enzymes include, but are not limited to: Oxidoreductase (EC 1.1.-.-); Malate oxidase (EC 1.1.3.3); Glucose oxidase (EC 1.1.3.4); Hexose oxidase (EC 1.1.3.5); L-gulonolactose oxidase (EC 1.1.3.8); Galactose oxidase (EC 1.1.3.9); Pyranose oxidase (EC 1.1.3.10); Xanthine oxidase (EC1.1.3.22); N-Acylhexosamine oxidase (EC 1.1.3.29); D-Arabinono-1,4-lactose oxidase (EC 1.1.3.37); Lactoperoxidase (EC 1.11.1-); Myeloperoxidase (EC 1.11.1.7); et al.

Yet another aspect of the invention consists of one or more anchor-enzyme complexes to degrade biofilm structures, which have been described previously, and a second component of one or more unbound or free non-enzymatic bactericidal components whose function is to kill newly exposed bacteria as the biofilm structure is removed. The non-enzymatic bactericidal agents include, but are not limited to, antimicrobial peptides, synthetic antimicrobial agents, antibiotics, sanitizing agents and host immune response elements.

The purpose of these various embodiments is to hold or retain the biofilm-degrading enzymes and bactericidal components in fluid-flow systems that are open, partially open or, at least not completely closed systems. Without the capability to keep the appropriate active agents at or near the biofilm structure, they may be swept away in the fluid flow.

Antibacterial and antifungal peptides have therapeutic value against microbial (bacteria and fungi) infections and in the treatment of cancer. These antimicrobial peptides show promise for treating topical infections, including those in the oral cavity. Porphyromonas gingivalis and Prevotella intermedia show differential sensitivity toward Cecropin B than commensal species (Devine, D. A., March, P. D., Percival, R. S., Rangarajan, M. and Curtis, M. S. “Modulation of Antibacgterial Peptide Activity by Products of Porphyromonas gingivalis and Prevotella spp.”. Microbiology, 145, 965-971; 1999). Retention on surfaces, such as skin, tissue in the oral cavity, vaginal tract, veins and arteries, etc, is difficult, if not impossible to achieve. However, the ability to retain the antibacterial/antifungal peptide at the desired site is substantially increased if the peptide is fitted with or connected to an anchor moiety or molecule.

Creating the anchored antibacterial/antimicrobial peptide can be achieved either through a recombinant protein using standard genetic engineering techniques or by chemical coupling reactions. For the purpose of illustration and not restricting the invention, a fusion protein can be used to treat subgingival infections which are the consequences, to a large measure, caused by Porphyromonas gingivalis.

Examples of selected members of classes of antimicrobial peptides are listed, not to restrict the invention, but rather to demonstrate the breadth of the application:

Generic Groups of Antimicrobial Peptides

Endolysin, cationic peptides, polymyxin B, protamine, bactenoicin, bacteriocin, lysine, protegrins, defensins, nisin, lacticin, BPI (bactericidal/permeability increasing), β-peptides, drosomycin and attacin. Other specific examples of antimicrobial peptides include Brevinin, CAMEL, Cecropin B, Magainin II, Mastoparan, Macrocyclic, Kalata, Cirulin-(A and B), cyclopsychotride, Mytilin (B, C, D and G1) and Seminal Plasmin SLS Fragment.

Representative examples of mammalian antimicrobial peptides:

| Peptide | Class |
|---|---|
| HNP-1 (α-defensin) | β-sheet |
| HBD-2 (β-defensin) | β-sheet |
| Protegrin | β-sheet |
| Indolicidin | Extended |
| Bac5 | Extended |
| Bactenicin | Loop (cyclic) |
| LL37 | α-helical |
| Cecropin P1 | α-helical |
| Macrocyclic | cysteine-knot |

### BRIEF DESCRIPTION OF THE DRAWINGS

FIG. 1 is a schematic view of a biofilm from a distance;

FIG. 2 is a schematic view showing the elements of a single layer within a biofilm structure;

FIG. 3 is a schematic view of a magnified section of a single biofilm layer; and

FIG. 4 is a diagram of a Robbins-type flow cell to measure biofilm dynamics under various flow conditions and components that may be added to the flowing fluid.

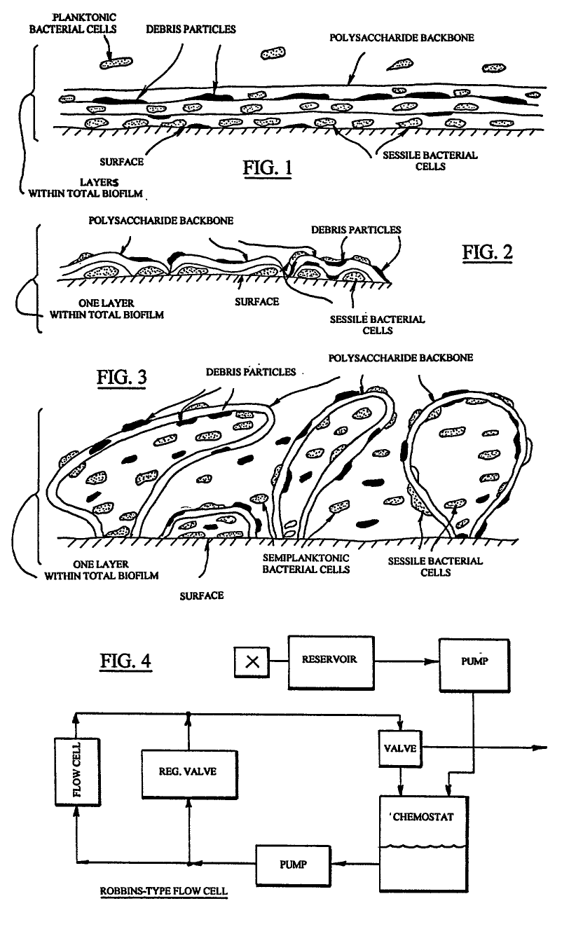

### DETAILED DESCRIPTION OF THE INVENTION

Pseudomonas aeruginosa is used as a preferred example in this description and was selected as an example because it produces a biofilm in a wide variety of conditions and circumstances. It is also associated with the genetic-based disease of cystic fibrosis. Pseudomonas aeruginosa also produces its bioflim in various industrial settings where water flow is part of the industrial processing. However, the principles described in this invention apply to all biofilms, independent of the causative organism producing the biofilm structure.

Pseudomonas aeruginosa, which is a gram-negative rod, is one of many organisms found in slime residues associated with a wide variety of industrial, commercial and processing operations such as sewerage discharges, re-circulating water systems (cooling tower, air conditioning systems etc.), water condensate collections, paper pulping operations and, in general, any water bearing, handling, processing, collection etc. systems. Just as biofilms are ubiquitous in water handling systems, it is not surprising that Pseudomonas aeruginosa is also found in association with these biofilms. In many cases, Pseudomonas aeruginosa is the major microbial component.

In addition to its importance in industrial processes, Pseudomonas aeruginosa and its associated biofilm structure has far-reaching medical implications, being the basis of many pathological conditions. Pseudomonas aeruginosa is an opportunistic bacterium that is associated with a wide variety of infections. It has the ability to grow at temperatures higher than many other bacteria and it is readily transferred from an environmental setting to become host-dependent. Translocation, both within a specific medium and to other media, is facilitated with its single polar flagella.

Pseudomonas aeruginosa has nutritional versatility in being able to use a wide variety of substrates, fast growth rate, motility, temperature resiliency and short incubation periods all of which contribute to it predominance in natural microflora communities as well as being the cause of nosocomial (hospital acquired) infections.

Infections caused by Pseudomonas aeruginosa begin usually with bacterial attachment to and colonization of mucosal and cutaneous tissues. The infection can proceed via extension to surrounding structures or infection may lead to bloodstream invasion, dissemination and sepsis syndrome.

Respiratory Infections: Alginate producing strains of Pseudomonas aeruginosa infect the lower respiratory tract of patients with cystic fibrosis leading to acute and the chronic progression of the pathological condition. The colonization of Pseudomonas aeruginosa accelerates disease pathology resulting in increased mucus production, airway obstruction, bronchiectasis and fibrosis in the lungs.

While cystic fibrosis is a chronic infection of Pseudomonan aeruginosa, other, acute, respiratory infections occur as a result of bacteria other than Pseudomonas aeruginosa. For example, Streptococcus pyrogenes is the primary cause of bacterial pharyngitis which, is uncontrolled, can lead to rheumatic fever. Nelson, et al. [Proc. Acad. Sci. 98, 4107-4112(2001)] report a lysis process to control the bacterial infection using double-stranded DNA bacteriophages. The enzymes associated with the bacteriophage-mediated lysis serve as examples of implementing the present invention.

Another example of implementing the present invention for acute respiratory infection caused by Streptococcus pneumonia entails the dismantling of the biofilm. Cartee, et al. [J. Biol. Chem. 275, 3907-3914(2000)] describe the synthesis of the Streptococcus pneumonia biofilm as being comprised of glycosidic linkages of the polysaccharide backbone. As an example of an enzyme anchor complex to dismantle the Streptococcus pneumonia biofilm would include the binding domain from β-glycosyltransferase (hyaluronic acid synthetase, chitin synthetase, cellulase synthetase, etc.) as the anchor and ex0-β-glucosidase as the enzyme.

Eye Infections: Pseudomonas aeruginosa colonization in the eye leads to bacterial keratitis or corneal ulcer and endophthalmitis.

Ear Infections: Pseudomonas aeruginosa is a common bacterium residing in the ear canal and is a common pathogen causing external otitis.

Urinary Tract Infections: Pseudomonas aeruginosa is a common causative agent in complicated and nosocomial urinary tract infections even though other bacterial species are present. Opportunities for infection occur during catheterization, surgery, obstruction and blood-borne transfer of Pseudomonas aeruginosa to the urinary tract.

Skin and Soft Tissue Infections: Pseudomonas aeruginosa can cause opportunistic infections in skin and soft tissue in locations where the integrity of the tissue is broken by trauma, burn injury, dermatitis and ulcers resulting from peripheral vascular disease. Dressings for these types of wounds, as well as wounds in general where an infection can develop, can incorporate the appropriate enzymes that would degrade initial biofilm formation on these dressings. Such systems are closed systems or mostly so, and consequently, the enzymes may or may not have moieties attached to them as a means of retaining them to the wound dressing. Further, an adjunct to the embodiment for this application there may also be associated with it suitable antimicrobial/antibiotic agents.

Endocarditis: Pseudomonas aeruginosa has been shown to have a high affinity to cardiac tissue including heart valve tissue.

Alginate Biofilms of Pseudomonas aeruginosa: At the root of Pseudomonas aeruginosa initial colonization, as well as its proliferative growth rate, is the production of a mucoid exopolysaccharide layer comprised of alginate. This exopolysaccharide layer, along with lipopolysaccharide, protects the organism from direct antibody and complement mediated bactericidal mechanisms and from opsonophagocytosis. This protective biofilm allows Pseudomonas aeruginosa to expand, grow and to exist in harsh environments that may exist outside the alginate biofilm.

The alginate biofilm or “slime matrix” consists of a secreted polysaccharide that serves as the backbone structure of the biofilm. Alginate is a polysaccharide copolymer of β-D-mannuronic acid and α-L-guluronic acid linked together by 1-4 linkages. The immediate precursor to the biosynthetic polymerization is guanosine 5′-diphosphate-mannuronic acid, which is converted to mannuronan. Post-polymerization of the mannuronan by acetylation at O-2 and O-3 and epimerization, principally at C-5, of some of the monomeric units to produce gulonate, results in varying degrees of acetylation and gulonate residues. Both the degree of acetylation and the percentage of mannuronic residues that have been converted to gulonate residues greatly affect the properties of the biofilm. For example, polymers rich in gulonate residues and in the presence of calcium, tend to be more rigid and stiff than polymers with low levels of gulonate monomeric units.

Construction of Anchor-Enzyme Complexes

The anchor enzyme complex of the invention can be constructed using chemical synthetic techniques. Additionally, the anchor-enzyme complex, if the anchor is a polypeptide or protein, such as protein binding domains, lectins, selecting, heparin binding domains etc., can be constructed using recombinant genetic engineering techniques.

Examples of Types of Anchors

The binding domain from elastase; Domains that bind to carbohydrates and polysaccharide; Lectins; Mannose Binding Lectin; Selectins; The binding domain from Heparin; The binding domains of Fibronectin; CD44 Protein

Type of enzymes

1. Generally, enzymes in the class EC 4.2.2._, which are polysaccharide lyases, which degrade the polysaccharide backbone structure of biofilms:

Glycoside Hydrolases, Galactoaminidases, Galactosidases, glucosaminidases, Glucosidases, Mannosidases (EC 3.1.2._); Neuraminidase (EC 3.1.2.18); Dextranase, Mutanase, Mucinase, Amylase, Fructanase, Galactosidase, Muramidase, Levanase, Neuraminidase (EC 3.2._); α-Glucosidases (EC 3.2.1.20); β-Glucosidase (EC 3.2.1.21); α-Glucosidase (EC 3.2.1.22); β-D-Mannosidase (EC 3.2.1.25); Acetylglucosaminidase (EC 3.2.1.30); Hyaluronoglucosaminidase (EC 3.2.1.35); α-L-Fucosidase (EC 3.2.1.51); Hyaluronate Lyase (EC 4.2.2.1); Pectate Lyase (EC 4.2.2.2); Alginate Lyase [Poly(/β-D-Mannuronate) Lyase] (EC 4.2.2.3); Chondroitin ABC Lyase (EC 4.2.2.4); Chondroitin AC Lyase (EC 4.2.2.5); Oligogalacturonide Lyase (EC 4.2.2.6); Heparin Lyase (EC 4.2.2.7); Heparan Lyase [Heparitin-Sulfate Lyase] (EC 4.2.2.8); Exopolygalacturonate Lyase (EC 4.2.2.9); Pectin Lyase (EC 4.2.2.10); Poly (α-L-Guluronate) Lyase (EC 4.2.2.11); Xanthan Lyase (EC 4.2.2.12); Exo-(1,4)-α-D-Glucan Lyase (EC 4.2.2.13); Non-specific polysaccharide depolymerases derived from bacteriophages et al.

2. Enzymes for removing debris embedded within the biofilm structure or extraneous byproducts as a result of removing the biofilm. This later debris may originate from the host and would include immune response products. These include many EC sub-classes with the general class of hydrolytic and digestive enzymes. In descriptive terms, they include enzymes that facilitate the breaking of chemical bonds and include the following:

Esterases—cleavage of ester bonds; Glycolytic—cleavage of bonds found in oligo—and polysaccharides; Peptidases-cleavage of peptide bonds where the substrate is a protein or polypeptide; Nucleic acid materials (RNA and DNA); Carbon-nitrogen cleavage—where the substrate is not a protein or polypeptide; Acid anhydride cleaving enzymes; Carbon-carbon bond cleavage; Halide bond cleavage; Phosphorus-nitrogen bond cleavage; Sulfur-nitrogen bond cleavage; and Carbon-phosphorus bond cleavage.

Typical Examples Include the Following Enzymes

Endopeptidases; Peptide Hydrolases (EC 3.4._) ; Aminopeptidases (EC 3.4.11); Nucleic Acid Hydrolases (EC 3.1.-.-); Propyl Aminopeptidases (EC 3.4.11.5); Glycylpropyl Dipeptidases; Dipeptidyl Peptidase (EC 3.4.14); Serine Endopeptidases (EC 3.4.21); Chymotrypsin (EC 3.4.21.1); Trypsin (EC 3.4.21.4); Amidohydrolases (EC 3.5._); N-Acetylglucosamine-6-phosphate Deacetylase (EC 3.5.1.25); Oxo-Acid Lyases (EC 4.1.3._); N-Acetylmuraminate Lyases (EC 4.1.3.3); Carbohydrate Epimerases (EC 5.1.3_); Glucosamine-6-phosphate Isomerases (EC 5.3.1.10).

Types of Bactericidal Agents

1. Enzymatic

A. Generation of Active Oxygen. Any member from the class of oxido-reductases, EC 1._that generate active oxygen; Monosasccharide oxidases, Peroxidases, Lactoperoxidases, Salivary peroxidases, Myeloperoxidases, Phenol oxidase, Cytochrome oxidase, Dioxygenases, Monooxygenases

B. Bacterial cell lytic enzymes. Lysozyme, Lactoferrin

2. Non-Enzymatic

A. Antimicrobial e.g., chlorhexidine, amine fluoride compounds, fluoride ions, hypochlorite, quaterinary ammonium compounds e.g. cetylpyridinium chloride, hydrogen peroxide, monochloramine, providone iodine, any recognized sanitizing agent or oxidative agent and biocides.

B. Antibiotics. Including, but not limited to the following classes and members within a class:

Aminoglycosides: Gentamicin, Tobramycin, Netilmicin, Amikacin, Kanamycin, Streptomycin, Neomycin;

Quinolones/Fluoroquinolones: Nalidixic Acid, Cinoxacin, Norfloxacin, Ciprofloxacin, Perfloxacin, Ofloxacin, Enoxacin, Fleroxacin, Levofloxacin;

Antipseudomonal: Carbenicillin, Carbenicillin Indanyl, Ticarcillin, Azlocillin, Mezlocillin, Piperacillin;

Cephalosporins: First Generation—Cephalothin, Cephaprin, Cephalexin, Cephradine, Cefadroxil, Cefazolin; Second Generation—Cefamandole, Cefoxitin, Cefaclor, Cefuroxime, Cefotetan, Ceforanide, Cefuroxine Axetil, Cefonicid; Third Generation—Cefotaxime, Moxalactam, Ceftizoxime, Ceftriaxone, Cefoperazone, Cftazidime;

Other Cephalosporins: Cephaloridine, Cefsulodin;

Other β-Lactam Antibiotics: Imipenem, Aztreonam;

β-Lactamase Inhibitors: Clavulanic Acid, Augmentin, Sulbactam;

Sulfonamides: Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole;

Urinary Tract Antiseptics: Methenamine, Nitrofurantoin, Phenazopyridine and other napthpyridines;

Penicillins: Penicillin G and Penicillin V;

Penicillinase Resistant: Methicillin, Nafcillin, Oxacillin, Cloxacillin, Dicloxacillin;

Penicillins for Gram_Negative/Amino penicillins: Ampicillin (Polymycin), Amoxicillin, Cyclacillin, Bacampicillin;

Tetracyclines: Tetracycline, Chlortetracycline, Demeclocycline, Methacycline, Doxycycline, Minocycline;

Other Antibiotics: Chloramphenicol (Chlormycetin), Erythromycin, Lincomycin, Clindamycin, Spectinomycin, Polymyxin B (Colistin), Vancomycin, Bacitracin;

Tuberculosis Drugs: Isoniazid, Rifampin, Ethambutol, Pyrazinamide, Ethinoamide, Aminosalicylic Acid, Cycloserine;

Anti-Fungal Agents: Amphotericin B, Cyclosporine, Flucytosine;

Imidazoles and Triazoles: Ketoconazole, Miconazaole, Itraconazole, Fluconazole, Griseofulvin;

Topical Anti Fungal Agents: Clotrimazole, Econazole, Miconazole, Terconazole, Butoconazole, Oxiconazole, Sulconazole, Ciclopirox Olamine, Haloprogin, Tolnaftate, Naftifine, Polyene, Amphotericin B, Natamycin.

### EXAMPLE

Since Pseudomonas aeruginosa is a ubiquitous bacterial strain, found not only in the environment and in industrial settings where fouling occurs, but also in many disease conditions, it will serve as an example to illustrate the principles of the invention. Further, while there are many disease conditions for which Pseudomonas aeruginosa is the cause, ocular infections will exemplify the implementation of the invention. The choice of Pseudomonas aeruginosa as the biofilm-producing bacteria and pathogen and ocular infection as a consequence of the biofilm is not meant to preclude or limit the scope of this invention. The principles outlined in this example readily apply to all biofilms, whether produced by bacteria or other organisms, all biofilms that are generated by organisms and the embodiments, taken and implemented either individually or collectively.

Pseudomonas aeruginosa is an opportunistic bacterial species, which once colonized at a site such as ocular tissue, produces a biofilm with a polysaccharide-based alginate polymer. This exopolysaccharide or glycocalyx matrix is the confine in which the bacterial species can grow and proliferate. This biofilm matrix can also serve as a medium for other, pathogenic bacteria, fungi and viruses. It is of therapeutic benefit, therefore, to remove the biofilm structure and eliminate all bacteria at the site, not only Pseudomonas aeruginosa.

Alginate lyase, the expression product from the algL gene, can be obtained from various bacterial sources e.g. Azotobacter vinelandii, Pseudomonas syringe, Pseudomonas aeruginosa etc., producing an enzyme AlgL, which degrades alginate. Other genes, e.g. alxM, also provide a wide variety of alginate lyase and polysaccharide depolymerase enzymes with degrade alginate by various mechanisms.

Endogenous lectins, heparin binding domains and various receptors from animals and plants have receptors that bind to alginate. These receptors, when located on host cell surfaces, allow the evolving alginate biofilm to be retained by the infected tissue. Elastase (Leukocyte Elastase, EC 3.4.21.37 and Pancreatic Elastase, EC 3.4.21.36), which is a digestive enzyme, also has a domain that binds to alginate. Such binding capability, along with the degradative ability of the catalytic site in elastase, has been implicated in tissue degradation associated with alginate biofilm infections such as cystic fibrosis. In addition, other serine proteases also have alginate binding domains.

In one aspect of the invention, a fusion protein is created, using standard genetic engineering techniques. One of the traits or elements of the fusion protein is the ability to degrade alginate and a second property being a binding capability of the newly-created fusion protein, derived from, for example, the binding domain of elastase. he bi-functional protein fulfills the criteria set out in the invention in that the binding domain derived from elastase serves as the anchor and the alginate lyase portion of the fusion protein serves as the degradative enzyme for the biofilm.

This embodiment can be used to degrade alginate-based biofilms in industrial processes where fouling occurs, or implanted medical devices, including catheters and cannulae. This embodiment can also be used for a wide variety of infections such as: ophthalmic applications (infections, implants, contact lenses, surgical manipulations etc.), respiratory infections, including pneumonia and cystic fibrosis, ear infections, urinary tract infections, skin and soft tissue infections, infections that occur in burn victims, endocarditis, vaginal infections, gastrointestinal tract infections where biofilms, either impair function or cause infections and in disease conditions, such as cystic fibrosis.

It is within the scope of this invention that the principles outlined here also apply to all biofilms in all circumstances in which they occur.

Construction of the Enzyme Anchor Complex

Using molecular biology and biotechnology techniques, gene fusions are created to produce unique proteins from recombinant DNA segments. A DNA sequence which specifically codes for an enzyme is fused to a DNA segment that specifically codes for a protein binding domain. The resulting fused DNA segment will produce a unique protein that possesses both enzymatic or catalytic activity and binding activity.

The DNA sequence that codes for alginate lyase obtained from Pseudomonas aeruginosa, or another acceptable strain, was isolated and amplified using polymerase chain reaction. The sequence was subcloned into an expression vector. Next the DNA that codes for leukocyte elastase was isolated from a mouse complementary DNA (cDNA) library. The mouse leukocyte elastase sequence was amplified by using polymerase chain reaction.

Both DNA sequences for alginate lyase and mouse leukocyte elastase were subcloned into a single open reading frame within a suitable expression vector. Thus, yielding a DNA sequence that codes for a single protein that contains both the amino acid sequence for alginate lyase as well as the sequence for leukocyte elastase. This hybrid or chimeric protein has the catalytic ability to degrade alginate as well as the binding ability of elastase.

Assay Procedure for Synthesized Anchor Enzyme Complexes

Preparation of Bacterial Biofilms. There are many procedures to prepare bacterial biofilms. Herein is one of those procedures.

The appropriate bacterial strain, or mixed strains if more than one strain is used, is incubated in tryptic soy broth for 18 to 24 hours at 37° C. After the incubation period, the cells are washed three times with isotonic saline and re-suspended in isotonic saline to a density of 106 CFU/ml. The re-suspended cells are incubated a second time with Teflon squares (1 ×1 cm) with a thickness of 0.3 cm for six to seven days at 37° C. The recovered cells in the saline incubation medium are planktonic bacteria, while those associated with the Teflon squares and the biofilm are sessile cells.

The biofilm-associated sessile cells are then treated with appropriate anchor-enzyme complexes that degrade the generated biofilm at various concentrations with or without bactericidal agents in either a completely closed system or an open system (flow-through chamber or cell). The bactericidal agent can be either an anchor enzyme system that generates active oxygen or a non-enzymatic, chemical that is a recognized antimicrobial agent, biocide or antibiotic.

Analysis of a Completely Closed System. The Teflon squares with the associated biofilm are transferred to isotonic saline medium containing a given concentration of anchor-enzyme complex that degrades the biofilm. At intervals of 3, 6, 12, 24 and 48 hours, the individual Teflon squares are washed three times with isotonic saline and finally added to fresh isotonic saline which is vigorously shaken or sonicated for tow minutes. The suspended mixture is diluted and counted for cell density and expressed as number of CFU/ml.

The same counting procedure can be used for the incubation medium.

Bactericidal agents are also incorporated into the experimental design, which also uses the same cell counting procedure.

Estimating Biofilm Size. At the end of any of the incubation steps, the biofilm can be recovered, dehydrated and weighed to obtain total biomass of the biofilm. Alternatively, the amount of alginate backbone can be determined where the biofilm contains Pseudomonas sp.

Extraction of Polysaccharide Backbone. After the second incubation and disruption of the biofilm, the bacterial cells are removed from the dispersion. With an increasing concentration of an ethanol/soling gradient, the alginate is precipitated, collected and washed three times with 95% ethanol. The precipitate is desiccated after which the quantity can be determined gravimetrically or by any number of chemical, enzymatic or combination of chemical and enzymatic methods. The most widely used method is the chemical method of which there are three types: uronic acid assay, orcinol-FeC13 and decarboxylation and C02 measurement.

Analysis in an Open System (Complete or Partial). The most widely used dynamic flow system that can be regulated from a completely closed to a completely open system is the Robbins Device or the Modified Robbins Device. The Modified Robbins Device allows the assessment of biofilms in which the fluid flow and growth rates of the biofilm can be regulated independently and simultaneously. A Robbins-type flow cell can be a completely closed system that possesses flow dynamics for assessing efficacy of anchor-enzyme complexes.

1. A composition for treating a biofilm structure including a cellular colony and the sessile cells associated with the biofilm structure, the composition comprising:
an enzyme selected for its ability to dismantle the biofilm structure;
an anchor molecule coupled to the enzyme to form an enzyme-anchor complex, the anchor molecule being capable of attaching to a surface on or proximal the biofilm structure, the anchor molecule being selected for its ability to bind to the cellular colony or other bioadhesive molecules;
wherein the attachment of the anchor to the surface permits prolonged retention time of the enzyme-anchor complex where the cellular colony and biofilm are present.

2. A composition as claimed in claim 1 wherein the enzyme is selected for its ability to degrade a living cellular colonizing matrix.

3. A composition as claimed in claim 1 wherein the enzyme-anchor complex is a fusion protein.

4. A composition as claimed in claim 1 when used for treating the biofilm associated with infections selected from the following group: ocular, contact lenses, cystic fibrosis, an implanted device, dermal infections, oral plaque.

5. A composition as claimed in claim 1 for treating the biofilm associated with industrial equipment and water handling systems.

6. A composition for treating a biofilm structure comprising:
a first enzyme-anchor component comprising an enzyme selected for its ability to degrade the biofilm structure and an anchor selected for its ability to attach to a surface on or proximal the biofilm structure to increase retention time, and
a second enzyme-anchor component comprising an enzyme selected for its ability to act directly upon bacteria from the biofilm structure for a bactericidal effect thereon and an anchor selected for its ability to attach to a surface on or proximal the biofilm structure.

7. A composition as claimed in claim 6 wherein the anchor of the first enzyme-anchor component and the anchor of the second enzyme-anchor component are the same.

8. A composition as claimed in claim 6 wherein the first enzyme-anchor component contains alginate lyase to degrade the biofilm structure.

9. A composition as claimed in claim 6 wherein the first enzyme-anchor component contains an alginate binding domain.

10. A composition as claimed in claim 9 wherein the alginate binding domain is derived from elastase.

11. A composition as claimed in claim 6 wherein first enzyme-anchor component is a fusion protein.

12. A composition as claimed in claim 6 wherein second enzyme-anchor component is a fusion protein.

13. A composition as claimed in claim 6 wherein the second enzyme-anchor component contains a cell wall degrading enzyme.

14. A composition as claimed in claim 13 wherein the cell wall degrading enzyme is selected from the group consisting of: a lysozyme to lyse bacteria within the biofilm, lactoferrin, lysin, endolysin and holin.

15. A composition as claimed in claim 6 wherein the second enzyme-anchor component comprises one or more from the group consisting of: oxido-reductase enzymes, peroxidase enzyme, hexose oxidase, lactoperoxidase and myeloperoxidase, for generating active oxygen for the purpose of killing bacteria within the biofilm.

16. A composition as claimed in claim 6 wherein the enzyme for the first enzyme-anchor component is selected from the group consisting of: carboxylic ester hydrolases, sulfuric ester hydrolases, glycosidases and lyases acting on polysaccharides

17. A composition as claimed in claim 7 wherein the anchor is selected from the group consisting of: concanavalin A, wheat germ agglutinin, other lectins, elastase, amylose binding protein, binding domains from enzymes, dextransucrase, starch-synthesizing enzymes, cellulose-synthesizing enzymes, chitin-synthesizing enzymes, glycogen-synthesizing enzymes, pectate synthetase, glycosyl transferase-binding domains (glucan-, mutan-, levan-, polygalactosyl-synthesizing enzymes).

18. A composition as claimed in claim 7 wherein the anchor is a disclosing agent for oral bacterial biofilms.

19. An ophthalmic composition for treating contact lenses for the eye comprising of a composition as claimed in claim 2.

20. A composition as claimed in claim 2 wherein enzyme-anchor complex is a fusion protein whose anchor molecule comprises an alginate-binding domain and whose enzyme is an alginate degrading enzyme.

21. An ophthalmic composition for treating ocular related infections comprising:
an enzyme-anchor complex having an enzyme component to degrade biofilm associated with the infection and an anchor componment for attachment at the biofilm to increase retention time, and
a bactericidal agent to kill individual bacteria that are released from the biofilm structure as it is being degraded.

22. A composition as claimed in claim 21 wherein the bactericidal agent is selected from the group consisting of: aminoglycoside antibiotic; a quinolone or fluoroquinolone antibiotic; a cephalosporin antibiotic; a penicillin antibiotic; and tobramycin.

23. A composition as claimed in claim 21 wherein the bactericidal agent is selected from the group consisting of: ciprofloxacin, ofloxacin, aztreonam, vancomycin, streptomycin, neomycin, and gentamicin.

24. A composition as claimed in claim 21 wherein the bactericidal agent is an antimicrobial peptide.

25. A composition as claimed in claim 21 wherein the bactericidal agent has an anchor.

26. A composition as claimed in claim 24 wherein the antimicrobial peptide has an anchor.

27. A composition as claimed in claim 21 wherein the anchor is selected from the group consisting of a polysaccharide binding domain and a cellulose binding domain.

28. A composition as claimed in claim 21 wherein the anchor is a binding domain selected from the group consisting of β-glycosyltransferase and an enzyme that is an exo-β-glucosidase.

29. A two component composition for treating a biofilm structure comprising, as the first component, an enzyme-anchor complex to degrade the biofilm structure and, as the second component, an antibacterial peptide coupled to an anchor and having the capability to act directly upon the bacteria for a bactericidal or fungicidal effect.

30. A composition as claimed in claim 29 wherein the antibacterial peptide is an bacteriocin.

31. A composition as claimed in claim 1 wherein the enzyme and anchor are selected to treat cystic fibrosis.

32. A two component composition comprising an enzyme-anchor complex to degrade biofilm structures and produce debris and a second enzyme-anchor complex having the capability to act upon debris.

33. A composition as claimed in claim 32 wherein the second enzyme has the capability to act on DNA.

34. A composition as claimed in claim 33 wherein the second enzyme is DNAse.

35. A method for the treatment of a biofilm structure comprising introducing to the biofilm structure an enzyme-anchor complex having an enzyme component to degrade the biofilm structure and an anchor component for attachment at the biofilm structure, and a bactericidal agent to kill individual bacteria that are released from the biofilm structure as it is being degraded.
